(19) United States
(12) Patent Application Publication
(10) Pub. No.: US 2017/0340434 A1
(43) Pub. Date: Nov. 30, 2017

(54) DEVICE USEFUL FOR LOCALIZED THERAPEUTIC DELIVERY WITHOUT FLOW OBSTRUCTION

(71) Applicant: Cook Medical Technologies LLC, Bloomington, IN (US)

(72) Inventors: Alec E. Cerchiari, Bloomington, IN (US); Gary L. Neff, Bloomington, IN (US); Brent A. Mayle, Spencer, IN (US); James C. Merk, Terre Haute, IN (US); Sean D. Chambers, Bloomington, IN (US); Ram H. Paul, JR., Bloomington, IN (US); Seoggwan Kim, West Lafayette, IN (US); Richard A. Swift, South Bend, IN (US)

(21) Appl. No.: 15/603,122

(22) Filed: May 23, 2017

Related U.S. Application Data
(60) Provisional application No. 62/340,858, filed on May 24, 2016.

(51) Int. Cl. A61F 2/07; A61M 31/00; A61B 17/12; A61M 25/00; A61F 2/966

(52) U.S. Cl. CPC A61F 2/07; A61M 25/002; A61F 2/966; A61B 17/12118; A61M 31/005; A61M 31/00; A61M 25/0026; A61F 2002/077; A61F 2250/0067; A61M 2025/0037

(57) ABSTRACT

Medical devices and methods are provided. In some aspects, devices useful for applying therapy locally within a body vessel are disclosed, the devices having a stent graft with flared end regions with a catheter providing fluid communication to the outer side of the narrower, intermediate region of the stent graft. Kits and systems including the same devices and methods are also disclosed.

### REFERENCE TO RELATED APPLICATIONS

This application is a non-provisional application claiming the benefit of U.S. Provisional Patent Application No. 62/340,858, filed May 24, 2016, pending, which is incorporated herein by reference.

### BACKGROUND

Many medical conditions are satisfactorily treated by the general systemic administration of a therapeutic agent. One drawback, however, associated with the systemic administration of therapeutic agents is that the systemically administered therapeutic agent may be absorbed not only by the tissues at the target site but by other areas of the body. Therefore areas of the body not needing treatment are also affected. Devices and methods for delivery of a therapeutic agent to only a selected portion of internal body tissue, without delivering the therapeutic agent to surrounding tissue or requiring additional systemic delivery of the therapeutic agent, are desired.

Medical delivery catheters provide a minimally invasive means for delivering therapeutic agents to internal body tissue. To provide site-specific localized treatment, balloon catheters may be used to deliver the therapeutic agent exclusively to the target site within a body vessel. One example of a condition that is beneficially treated by local administration of the therapeutic agent with a balloon catheter is the delivery of the therapeutic agent in combination with percutaneous transluminal coronary angioplasty (PTCA). Local administration of a therapeutic agent, however, is also beneficial in targeted chemotherapy, focal occlusion of vessels, thrombolysis, and targeted cell delivery, just to name a few examples.

During a site-specific therapy using existing balloon catheters, the catheter balloon is positioned at a target site, and the balloon is inflated, filling the vessel. The balloon is subsequently deflated and the catheter removed from the target site and from the patient's lumen thereby to allow fluid (e.g., body fluid) to flow freely through the lumen. Unfortunately, however, these balloon-based systems occlude blood flow and often cause distal ischemia, thereby limiting the window of time available for therapeutic delivery and their target sites for deployment. Therefore, there is a need for a tool that can deliver therapeutics, contrast agents, or biologics to a specific site within the human body without causing fluidic or gaseous occlusions. Perfusion balloon catheters exist; however, many allow only a small percentage of perfusion. For example, a balloon catheter sized for a 6 mm body vessel may only provide a 1 mm passageway.

In view of current devices and methods, there is a need for a medical device for applying vascular therapy locally within a body vessel while allowing fluid flow to areas distal to the treatment site.

### SUMMARY

Medical devices and methods are provided. In some aspects, the present disclosure provides devices useful for applying therapy locally within a body lumen, such as a vascular vessel including arteries or veins while allowing flow through the vessel, the devices having a stent graft with flared end regions (e.g., a dog-bone stent graft) with a catheter providing fluid communication to the outer surface of the narrower, intermediate region of the stent graft. Specifically, in certain embodiments, the devices comprise a stent graft extending from a proximal end region to a distal end region and having an intermediate region positioned intermediate the proximal end region and the distal end region; the stent graft configurable from a contracted configuration to an expanded configuration; the stent graft defining a central lumen extending from the proximal end region to the distal end region in the expanded configuration and having a graft material portion in the intermediate region; the graft material portion having an inward-facing side that faces towards the central lumen and an outward-facing side that faces away from the central lumen; the proximal end region and the distal end region each having an average outer dimension when the stent graft is in the expanded configuration; the average outer dimensions, in the expanded configuration, of the proximal end region and the distal end region each being greater than an average outer dimension defined by the graft material of the intermediate region in the expanded configuration; and at least a first infusion/aspiration port in fluid communication with at least a first infusion/aspiration lumen; the first infusion/aspiration port opening on the outward-facing side of the graft material and the first infusion/aspiration lumen extending proximally from the first infusion/aspiration port along the proximal end region of the stent graft.

The proximal end region and/or the distal end region can be arranged to contact an inner surface of the body lumen wall to fluidly seal the intermediate region from other regions of the body lumen. The first infusion/aspiration lumen is preferably fluidly isolated from the central lumen. The stent graft can include a stent having an outward-facing side that faces away from the central lumen of the stent graft and an inward-facing side that faces towards the central lumen; and wherein the first infusion/aspiration lumen extends along the outward-facing side of the stent in the proximal end region. In some instances, a second infusion/aspiration port is in fluid communication with a second infusion/aspiration lumen; and the second infusion/aspiration port opens to the outward-facing of the graft material. In such instances, the first infusion/aspiration port and the second infusion/aspiration port can be diametrically and/or longitudinally opposed relative to the intermediate region of the stent graft. The first infusion/aspiration lumen and/or second infusion/aspiration lumen can be defined by one or more catheters, such as a bifurcated catheter. The infusion/aspiration lumens may have one or more infusion/aspiration ports per infusion/aspiration lumen.

The infusion/aspiration lumen can be positioned between the graft material and a second graft material with the second material bonded to the graft material. In some arrangements, the first infusion/aspiration extends proximally through a sheath and the sheath is sized and configured to contain the stent graft in the contracted configuration.

The average outer dimensions in the expanded configuration can be the average outer dimensions when the stent graft is expanded in its unconstrained condition. And, the proximal end region, the intermediate region, and the distal end region can each have an average outer dimension in the contracted configuration, and the average outer dimension of the proximal end region, the intermediate region, and/or the distal end region in the expanded configuration can be at least 20% greater than the average outer dimension of the same region in the contracted configuration. Preferably, the average outer dimension the region of highest expansion is at least 20% greater in the expanded configuration than in the contracted configuration. The proximal end region of stent graft can include a transitional portion and the transitional portion can have a portion positioned along a central, longitudinal axis of the stent graft when the stent graft is in an expanded configuration. In some instances, the transitional portion includes a helically-extending material (e.g., a spiral cut cannula).

Methods of using the devices of the present disclosure can include infusing a therapeutic agent through the first infusion/aspiration lumen towards the first infusion/aspiration port and out of the first infusion/aspiration port into contact with the outward-facing side of the graft material. Methods may include drawing a fluid contacting the outward-facing side of the graft material through the first infusion/aspiration port and into the first infusion/aspiration lumen. And, in some instances, methods include drawing a fluid contacting the outward-facing side of the graft material through a second infusion/aspiration port and into a second infusion/aspiration lumen; wherein the second infusion/aspiration port opens to the outward-facing side of the graft material. Advantageously, such arrangements can allow for continuous perfusion and/or aspiration of therapeutic agent. Additionally, such arrangements can help maintain high therapeutic concentrations and/or remove undesired side-products.

Methods of using the devices disclosed herein also include infusing a first liquid out of an infusion/aspiration port and into contact with the outward-facing side of the graft material and the first liquid transitioning into hydrogel form. In some instances, methods include infusing a first liquid out of an infusion/aspiration port and into contact with the outward-facing side of the graft material and infusing a second liquid out of an infusion/aspiration port and into contact with the first liquid so as to combine the first and second liquids. Such an arrangement may be useful for the combination of liquids inside a vessel of a patient, without the liquids reacting to one another or to the body of the patient prior to being exposed to the outward-facing side of the graft material.

The first and/or second liquids may include chemicals which combine to form a hydrogel in vivo for sealing or for regenerative applications. The following substances are contemplated as being infused and, when in contact with the outward-facing side for the graft material, transitioning from soluble liquid to hydrogel: hyaluronic acid, chitosan, alginate, poly(ethylene glycol), poly(N-isopropyl-acrylamide), proteins (e.g., collagen or fibrinogen), and/or peptides (e.g., RGD or IKVAV).

The transition from soluble liquid to hydrogel can occur spontaneously (i.e., physical self-assembly), through the action of chemical crosslinkers (i.e., catalyzed polymerization), and/or through irradiation (e.g., UV light irradiation). Self-assembly can take place through physical interactions that occur in response to temperature changes (e.g., from room temperature to body temperature) or time-sensitive changes in ionic and/or hydrophobic interactions. If hydrogel-formation occurs chemically, the liquid precursor reacts with another substance (e.g., liquid) that enables polymerization and, therefore, transition from soluble liquid to hydrogel upon mixing. The following cross-linkers may be used for polymerization: azide-alkyne(s), thiol-ene(s), diels-alder(s), oxime(s), and/or biologic(s) (e.g., thrombin). For hydrogel-formation through irradiation, the liquid precursor is irradiated, in many instances by light (e.g., UV light), causing the liquid to transition into hydrogel. In such instances, the device may be equipped with one or more emitters (e.g., light emitting diodes or fiber optic guides) arranged to irradiate the precursor liquid.

In arrangements that include the infusion of a first liquid and the infusion of a second liquid, the first and second liquids may be infused through separate infusion/aspiration lumens and/or ports. For example, the first liquid may be infused through a first infusion/aspiration lumen and a first infusion/aspiration port and the second liquid infused through a second infusion/aspiration lumen and a second infusion/aspiration port. However, it is contemplated that first and second liquids, in some instances, may be infused through the same infusion/aspiration lumen and/or infusion/aspiration port.

The present disclosure also provides kits including a stent graft, a catheter, and a sheath within a sterily sealed package; wherein the stent graft has a proximal portion, a distal portion, an intermediate portion positioned intermediate the proximal portion and the distal portion, and a stent configurable from a contracted configuration to an expanded configuration; wherein the stent graft includes a graft material portion extending along the intermediate portion; wherein the proximal portion and the distal portion each have an average outer dimension greater than an average outer dimension defined by the graft material portion in the intermediate portion; wherein the catheter communicates with a first infusion/aspiration port through a first infusion/aspiration lumen; wherein the first infusion/aspiration port opens to an outer side of the graft material of the stent graft; and wherein the stent graft and the catheter are positioned within a lumen of the sheath with the stent in the contracted configuration. In some instances, the kit includes a therapeutic agent in a container within the sterily sealed package.

Devices comprising a stent graft defining a central lumen extending from a proximal end region to a distal end region, the stent graft having an intermediate region positioned intermediate the proximal end region and the distal end region; and a first infusion/aspiration lumen extending along an outward-facing side of a graft material of the stent graft in the proximal end region and communicating with a first infusion/aspiration port is positioned in the intermediate region are also contemplated. In some instances, the stent graft of such devices includes a dog-bone shaped stent.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a perspective view of a device of the present disclosure in an expanded configuration.

FIG. 2 is a side view of a device the present disclosure in an expanded configuration.

FIG. 3 is a side view of a device of FIG. 2 positioned within a vessel of the patient in the expanded configuration.

FIG. 4 is a cross-sectional view of a device of the present disclosure in an expanded configuration.

FIG. 5 is a cross-sectional view of a device in a contracted configuration in a delivery sheath.

FIG. 6 is a side view of a stent in a contracted configuration.

FIG. 7 is a side view of the stent of FIG. 6 in an expanded configuration.

FIG. 8 is a side view of a device positioned within a vessel of the patient in the expanded configuration.

FIG. 9 is a plan view of a device of the present disclosure.

FIG. 10 is a cross-sectional view taken along line 10-10 of FIG. 9.

FIG. 11 is a plan view of a kit of the present disclosure.

FIG. 12 is a cross-sectional view of a device of the present disclosure in an expanded configuration in a body vessel of a patient.

FIG. 13 is a side view of a device the present disclosure.

FIG. 14 is a side view of a device of the present disclosure.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail; although it will be apparent to those skilled in the relevant art that some features that are not relevant to the present invention may not be shown for the sake of clarity.

The language used in the claims and the written description and in the following definitions is to only have its plain and ordinary meaning, except for terms explicitly defined below. Such plain and ordinary meaning is defined here as inclusive of all consistent dictionary definitions from the most recently published (on the filing date of this document) general purpose Merriam-Webster dictionary.

As used in the claims and the specification, the following terms have the following defined meanings:

As used herein, the term “aspiration” means the withdrawal by suction. It includes but is not limited to the partial or complete removal of a material from a location of the body.

As used herein, the term “catheter” means an elongate medical device defining an internal lumen along a length thereof and configured for insertion into canals, vessels, passageways, or body cavities for diagnostic or therapeutic purposes. The term includes but is not limited to single-lumen and multi-lumen elongate medical devices having a length sufficient to extend outside of the body of the patient (e.g., 20 cm or longer) and that are useful for the infusion or aspiration of fluid.

As used herein, the term “cells” includes endothelial cells, mesenchymoangioblasts, and bioengineering immune cells. For example, the cells can be cardiac muscle cells, lung cells, mesentery cells, or adipose cells. The adipose cells may be from omental fat, properitoneal fat, perirenal fat, pericardial fat, subcutaneous fat, breast fat, or epididymal fat. In certain embodiments, the cells comprise stromal cells, stem cells, or combinations thereof. Additional illustrative cells which can be used include hepatocytes, epithelial cells, Kupffer cells, fibroblasts, neurons, cardiomyocytes, myocytes, chondrocytes, pancreatic acinar cells, islets of Langerhans, osteocytes, myoblasts, satellite cells, endothelial cells, adipocytes, preadipocytes, biliary epithelial cells, and progentior cells of any of these cell types. The cells can include endothelial progenitor cells (EPCs) and/or muscle derived cells, including muscle derived myoblasts and/or muscle derived stem cells. The muscle derived cells can express desmin, M-cadherin, MyoD, myogenin, CD34, and/or Bcl-2, and can lack expression of CD45 or c-Kit cell markers. The term “cells” includes cellular populations of different types of cells, including adipose-derived stem and regenerative cells, sometimes also referred to as stromal vascular fraction cells, which can be a mixed population including stem cells, endothelial progenitor cells, leukocytes, endothelial cells, and vascular smooth muscle cells, which can be adult-derived. The “cells” (e.g., cellular preparation) can include adipose-derived cells that can differentiate into a nerve cell or a muscle cell. Suitable such cells and methods for obtaining them are described for example in U.S. Pat. No. 6,777,231 and U.S. Pat. No. 7,595,043, each of which is hereby incorporated herein by reference in its entirety

As used herein, the term “endothelial progenitor cells” or “EPCs” include endothelial colony forming cells (ECFCs), especially ECFCs with high proliferative potential. Suitable such cells are described for example in U.S. Patent Application Publication No. 20050266556 published Dec. 1, 2005, publishing U.S. patent application Ser. No. 11/055,182 filed Feb. 9, 2005, and U.S. Patent Application Publication No. 20080025956 published Jan. 1, 2008, publishing U.S. patent application Ser. No. 11/837,999, filed Aug. 13, 2007, each of which is hereby incorporated by reference in its entirety. Such ECFC cells can be a clonal population, and/or can be obtained from umbilical cord blood of humans or other animals. The endothelial colony forming cells can have the following characteristics: (a) express the cell surface antigens CD31, CD105, CD146, and CD144; and/or (b) do not express CD45 and CD14; and/or (c) ingest acetylated LDL; and/or (d) replate into at least secondary colonies of at least 2000 cells when plated from a single cell; and/or (e) express high levels of telomerase, at least 34% of that expressed by HeLa cells; and/or (f) exhibit a nuclear to cytoplasmic ratio that is greater than 0.8; and/or (g) have cell diameters of less than about 22 microns. Any combination of some or all of these features (a)-(g) may characterize ECFCs used in the present disclosure.

As used herein, the term “fluid” means a substance capable of flow and includes gasses and liquids.

As used herein, the term “infusion” means the introduction into a location of the body. It includes but is not limited to the introduction of a solution or suspension and may include introducing such a material into a vessel of a patient.

As used herein, the term “outer dimension” means the distance between outer surfaces in a plane orthogonal to the longitudinal axis of the stent graft. The term includes distances measured along radii of the stent graft.

As used herein, the term “stem cells” is used in a broad sense and includes traditional stem cells, adipose derived stem cells (e.g., cells derived from adipose tissue), progenitor cells, preprogenitor cells, reserve cells, and the like. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Suitable such stem cells and methods for obtaining them are described, for example, in U.S. Pat. No. 6,866,842 and U.S. Pat. No. 7,155,417, each of which is hereby incorporated herein by reference in its entirety.

As used herein, the term “stent graft” means a device having a stent and a graft material (e.g., a covering material) extending along a portion of the stent. The stent graft may be tubular in shape. The stent may include a frame comprising or consisting of a biocompatible metal or metal alloy, such as stainless steel, nickel-titanium (e.g., Nitinol), gold, platinum, palladium, titanium, tantalum, tungsten, molybdenum, or alloys thereof. Other suitable alloys for the stent include cobalt-chromium alloys such as L-605, MP35N, and Elgiloy; nickel-chromium alloys, such as alloy 625; and niobium alloys, such as Nb-1% Zr, and others. Preferably, the stent is MRI-compatible and does not produce artifacts in images or scans obtained from magnetic resonance imaging. The stent may be fabricated from wire, tubing, or sheet using metal working and finishing techniques known in the art, such as drawing, extrusion, cold forming, gun drilling, laser welding, and laser cutting technologies. One or more of the stents of the stent graft may alternatively be made from a non-metallic material, such as a thermoplastic or other polymer. The stents may be designed to be either balloon-expandable or self-expanding. The material of the self-expanding stent preferably has shape memory/superelastic characteristics that enable it to “remember” and recover a previous shape. In the case of nickel-titanium shape memory alloys, the source of the shape recovery is a phase transformation between a lower temperature phase (martensite) and a higher temperature phase (austenite), which may be driven by a change in temperature (shape memory effect) and/or by the removal of an applied stress (superelastic effect). Strain introduced into the alloy in the martensitic phase to achieve a shape change may be substantially recovered upon completion of a reverse phase transformation to austenite, allowing the alloy to return to the previous shape. Recoverable strains of up to about 8-10% are generally achievable with nickel-titanium shape memory alloys. Other suitable shape memory alloys for the stent may include, for example, Cu—Zn—Al alloys and Fe—Ni—Al alloys. Polymer materials that may be suitable for the stent include polyether ether ketone (PEEK), polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxy-butyrate valerate or another biodegradable polymer. Biodegradable metals such as magenesium and magnesium alloys are also contemplated. The graft material is a biocompatible material suitable as a barrier between body fluid and therapeutic agent and is preferably liquid impermeable. The graft material may be elastic and/or inelastic and/or may form a sleeve (e.g., tubular body). The graft material may be a continuous, unitary sheet of material or may comprise separate pieces of material. The graft material may comprise a woven or nonwoven sheet. The graft material can be pulled over the stent(s) and secured to structural components of the stent(s) by sutures, by loops of graft material, and/or by bonding with other material layers. Many different types of natural or synthetic graft materials may be employed. For example, the graft material may be formed in whole or in part from one or more silicones or polyesters, such as poly(ethylene terephthalate) or Dacron®; fluorinated polymers, such as polytetrafluoroethylene (PTFE) and expanded PTFE; polyurethanes; polypropylene; polyaramids; polyacrylonitrile; and/or nylons. Graft materials that are not inherently biocompatible may be suitable for use in the stent graft if they can be rendered biocompatible by, for example, surface modification techniques. Examples of surface modification techniques include graft material polymerization of biocompatible polymers from the material surface, coating of the surface with a crosslinked biocompatible polymer, chemical modification with biocompatible functional groups, and immobilization of a compatibilizing agent, such as heparin or other substances. It is also envisioned that the graft material may be impregnated or coated with one or more therapeutic drugs for release at the site of the aneurysm. The stent graft, or portions thereof, may be biodegradable.

As used herein, the term “therapeutic agent” means a substance useful in the treatment of a disease or disorder. It includes, but is not limited to small molecule drugs and contrast agents, nanoparticles, macromolecules, and cells. The term includes small molecule drugs useful for localized chemotherapy/oncology and/or vascular intervention such as dissolving thrombus and/or reducing vascular calcification. For example, drugs such as paclitaxel, rapamycin, myotropic/neurotropic antispasmodics, and anticalcificants such as phosphate binders are included. Contrast agents suitable for MRI, X-Ray, and/or ultrasound imaging are included, such as gadolinium, manganese, iron oxide, and iodine-based (ionic/non-ionic) contrast agents. Organic, inorganic, and/or complex/polymeric nanoparticles useful for thermal ablation and targeted drug-delivery are contemplated. This includes but is not limited to liposomes, micelles, perfluorocarbons, gold nanoparticles, superparamagnetic iron oxide nanoparticles (SPION), dendrimers and functionalized nanoparticles. Macromolecule proteins, peptides, and/or synthetic polymers useful for biochemical thrombectomy, cell adhesion, coercive morphogenesis, prolonged drug-release, and/or sealants are contemplated. This includes but is not limited to fibrinolytics (e.g., urokinase, tPA), adhesional proteins (e.g., Fn, Lama, Col), growth factors (e.g., VEGF, TGF, Insulin), drug-eluting gels, hydrogels and glues. Environmentally-responsive hydrogels that can transition from liquid to gel form at a desired temperature (e.g., at 37° C.) and concentration are contemplated. Cells including differentiated, stem/progenitor, and/or genetically modified cells useful for re-endothelialization, endothelial regeneration and/or cellular therapy are contemplated as well as antisense and monoclonal antibodies.

Devices described herein include a hollow, dog-bone shaped, and self-expanding stent graft coupled with a catheter that allows for both infusion and aspiration (either simultaneously or independently) of a soluble cargo. Additionally, disclosed devices can be deployed within and subsequently retrieved from the target site. Advantageously, disclosed devices can isolate a segment of wall a body lumen, such as a vascular vessel, to locally deliver molecules or cells to the wall of the body lumen, an implanted medical device in the segment of the body lumen, and/or branch lumens of the body lumen without completely obstructing fluidic or gaseous flow through the body lumen.

It is contemplated that methods of using the devices disclosed herein may include adjusting the inflow and outflow of therapeutic agent into a target site based on the catalytic performance of the therapeutic agent in the target site. Additionally, it is contemplated that methods of using the devices disclosed herein to deliver cells may include adjusting the inflow of the suspension of cells and outflow of fluid (e.g., suspension of cells that did not adhere to the target site).

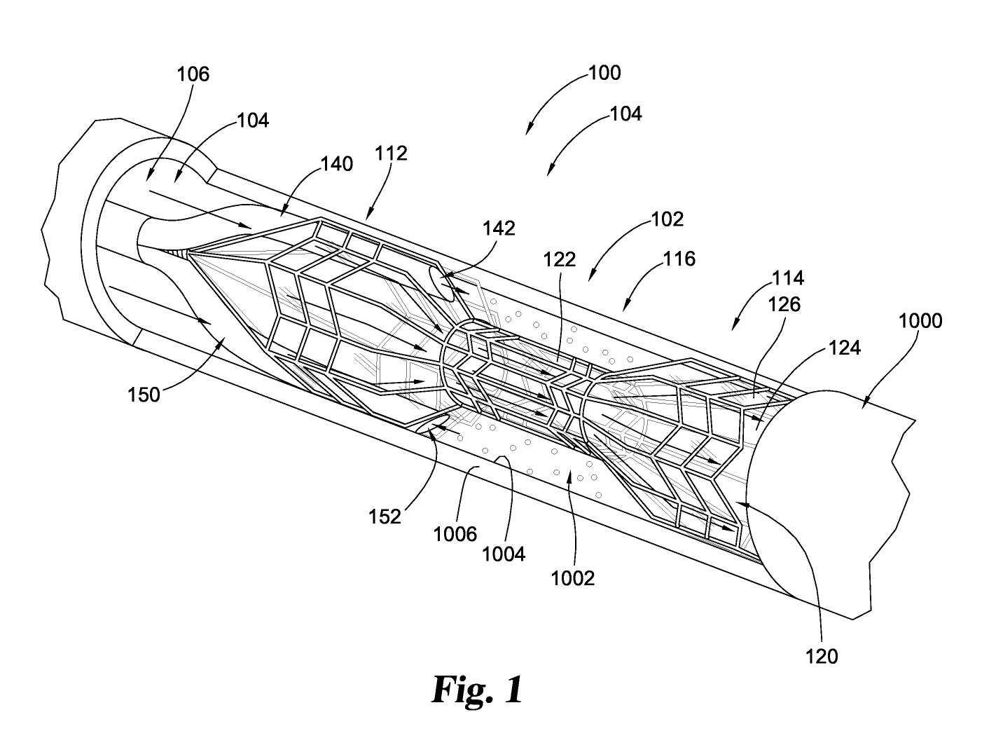

FIG. 1 illustrates a perspective view of an exemplary device 100 of the present disclosure. The device comprises a stent graft 102, a catheter 104, and a delivery sheath 106. The stent graft has a proximal end region 112, a distal end region 114, and an intermediate region 116 positioned intermediate the proximal end region and the distal end region.

The stent graft is configurable from a contracted configuration to an expanded configuration. In the expanded configuration, the stent graft defines a central lumen 120 extending along a length of the stent graft. The lumen is defined at least by a graft material 122 having an inward-facing side 124 that faces the central lumen 120 and an outward-facing side 126 that faces away from the central lumen 120 (i.e., towards the vessel wall when positioned within the body of a patient). The graft material has a portion positioned at least in the intermediate region of the stent graft and, in some instances, extends along the proximal end region and/or the distal end region of the stent graft. The graft material portion can be positioned on an inward-facing surface and/or an outward-facing surface of a stent of the stent graft.

The proximal end region, the intermediate region, and the distal end region each have an average outer dimension when the stent graft is in the expanded configuration. The expanded configuration may be the expanded configuration when the stent graft is expanded in its unconstrained condition (e.g., without constraint from a sheath or vasculature) or expanded within a sheath or vasculature of a patient. The average outer dimension 130 of the proximal end region and the average outer dimension 132 of the distal end region are each greater than the average outer dimension 134 of the intermediate region when the stent graft is in the expanded configuration. When the stent graft is positioned within a vessel 1000 of a patient, a space 1002 is defined between the intermediate region of the stent graft and the inner surface 1004 of the vessel wall 1006. In some instances, the space is an annular space, extending circumferentially around the intermediate region of the stent graft. The space may also be bounded at its proximal end by the proximal end region and at its distal end by the distal end region.

The catheter of the device defines and/or communicates with a first infusion/aspiration lumen 140 communicating with at least a first infusion/aspiration port 142. A portion of the first infusion/aspiration lumen (e.g., a portion of a catheter defining the infusion/aspiration lumen) extends along the proximal end region of the stent graft and positions the first infusion/aspiration port to open to the outward-facing surface of the graft material. In some instances, the first infusion/aspiration port is positioned along or adjacent to the intermediate region of the stent graft. The first infusion/aspiration port provides fluid communication between the first infusion/aspiration lumen and the space defined by the outward-facing surface of the graft material in the intermediate region of the stent graft and the inner surface of the vessel wall when the stent graft is positioned in the expanded configuration within a vessel.

In some instances, the catheter and/or first infusion/aspiration lumen are positioned on the inward-facing surface of the graft material portion in the proximal end region and extend through an opening in the graft material in the proximal end region or intermediate region to provide fluid communication between the first infusion/aspiration lumen and a space on the outward-facing surface of the first material. In some instances, however, the catheter and/or first infusion/aspiration lumen extend along a length of the proximal end region on the outward-facing surface of the graft material portion in the proximal end region of the stent graft.

In some instances, the catheter includes a second infusion/aspiration lumen 150 and at least a second infusion/aspiration port 152 in fluid communication with the second infusion/aspiration lumen Similar to the first infusion/aspiration lumen and first infusion/aspiration port, the second infusion/aspiration lumen can extend along the proximal end region of the stent graft and position the second infusion/aspiration port in fluid communication with the outward-facing surface of the graft material portion in the intermediate region.

The first infusion/aspiration port and/or second infusion/aspiration port may open in a direction parallel to the longitudinal axis of the stent graft or transverse to the longitudinal axis of the stent graft. For example, the infusion/aspiration port may be angled away from the longitudinal axis of the stent graft, so as to face a vessel wall when implanted. Additionally or alternatively, the first infusion/aspiration port and/or second infusion/aspiration port may be defined by a tapered end of the catheter.

In some arrangements, the catheter has a proximal end region and a distal end region and a bifurcation of the catheter in the distal end region forms the first infusion/aspiration lumen and the second infusion/aspiration lumen. In such instances, the first infusion/aspiration lumen and second infusion/aspiration lumen may be in fluid communication with a catheter lumen in the proximal end region of the catheter. However, in some instances, the first infusion/aspiration lumen and the second infusion/aspiration lumen may separately extend from the distal end region of the catheter to the proximal end region of the catheter without being in fluid communication with one another. As will be appreciated, the catheter may be a multi-lumen catheter and may include one or more additional lumens for infusion/aspiration and/or for receiving auxiliary devices such as a wire guide or microcatheter.

The first infusion/aspiration port, second infusion/aspiration port, first infusion/aspiration lumen, and/or second infusion/aspiration lumen may be spaced from another around the circumference of the intermediate region of the stent graft. For example, the first infusion/aspiration port and second infusion/aspiration port may be diametrically opposed relative to the intermediate region of the stent graft. Advantageously, such an arrangement may provide for even distribution of the therapeutic agent in the space defined by the intermediate region stent graft and the inner surface of the vessel wall. Additionally, in some instances, the first infusion/aspiration lumen and first infusion/aspiration port are useful for the infusion of fluid (e.g., therapeutic agent) into the space around the intermediate region of the stent graft (i.e., in fluid communication with the outward-facing surface of the graft material) and the second infusion/aspiration lumen and second infusion/aspiration port are useful for the withdrawal (e.g., aspiration) of fluid (e.g., blood and/or therapeutic agent) from the space defined by the intermediate region of the stent graft and the inner surface of the vessel wall, when the stent graft is positioned within a vessel.

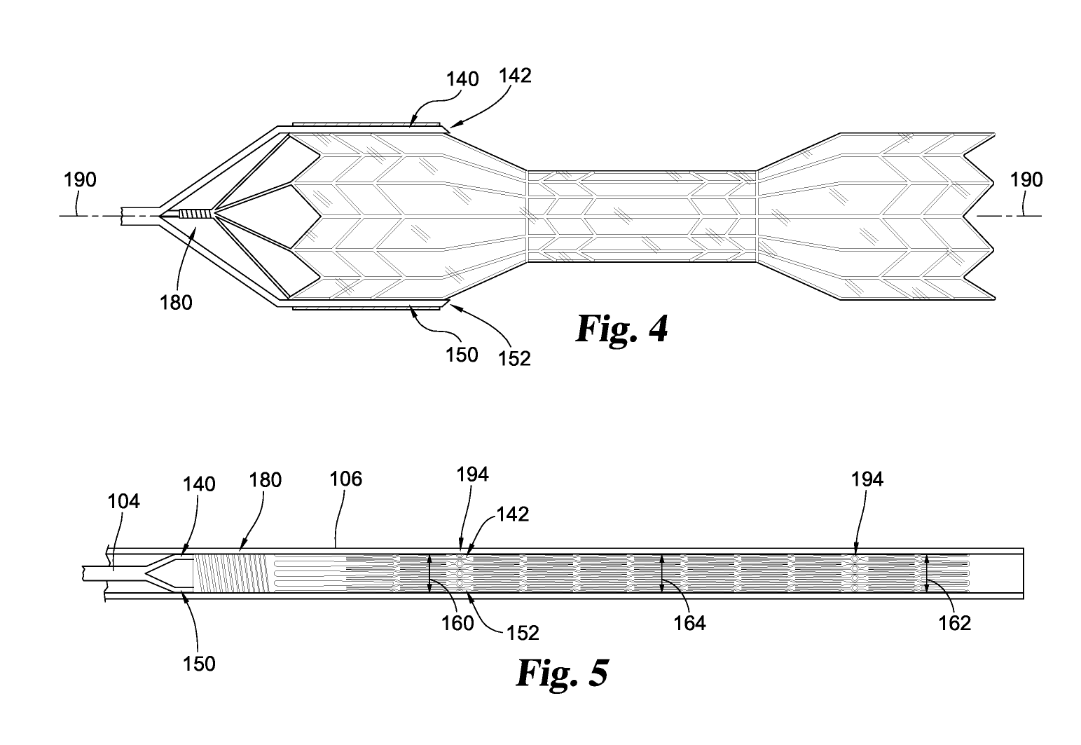

The catheter may be attached to the stent graft by one or more sutures, by bonding the catheter to the stent graft with a film and/or adhesive, and/or by molding a portion of the stent graft in the catheter. For example, as illustrated in FIG. 4, the catheter may be positioned between a first graft material and a second graft material with the second graft material bonded to the first graft material and sandwiching the catheter there between. In some instances, the catheter is detachably connected to the stent graft, with the catheter being capable of detachment from the stent graft under a force of <10N or even <5N when the stent graft is in the expanded configuration within the body of a patient. It is contemplated that the first infusion/aspiration lumen and/or the second infusion/aspiration lumen may be defined by opposing layers or plies of graft material extending along a length of the proximal end region of the stent graft. In such instances, the first infusion/aspiration lumen and/or the second infusion/aspiration lumen may communicate with one or more catheters extending proximally of the proximal end region of the stent graft. It is also contemplated that one or more infusion/aspiration lumens may be defined by a portion of the stent, such as a hollow strut.

In some arrangements, the device includes a sheath sized and configured to contain the stent graft in the contracted configuration. This sheath may be arranged for advancement through the vasculature of a patient, such as by including a hydrophilic coating and/or rounded distal tip. Portions of the catheter are also contained within the sheath when the stent graft is contained within the sheath in the contracted configuration.

In the contracted configuration, the proximal end region of the stent graft has an average outer dimension 160, the distal end region has an average outer dimension 162, and the intermediate region has an average outer dimension 164. In some instances, the average outer dimension of the proximal end region, the intermediate end region, and/or the distal end region in the expanded configuration is at least 20% greater than the average outer dimension of the same region in the contracted configuration in the region of highest expansion. Additionally or alternatively, the proximal end region, the intermediate region, and the distal end region each can have an average outer dimension in the contracted configuration of 7 mm or less.

Devices of the present invention may have multiple intermediate regions with the “intermediate region” referenced herein being one of the multiple intermediate regions. For example, any of the devices mentioned herein may include two or more intermediate regions, with at least one of the intermediate regions having a smaller outer dimension relative to one or more of the other intermediate regions.

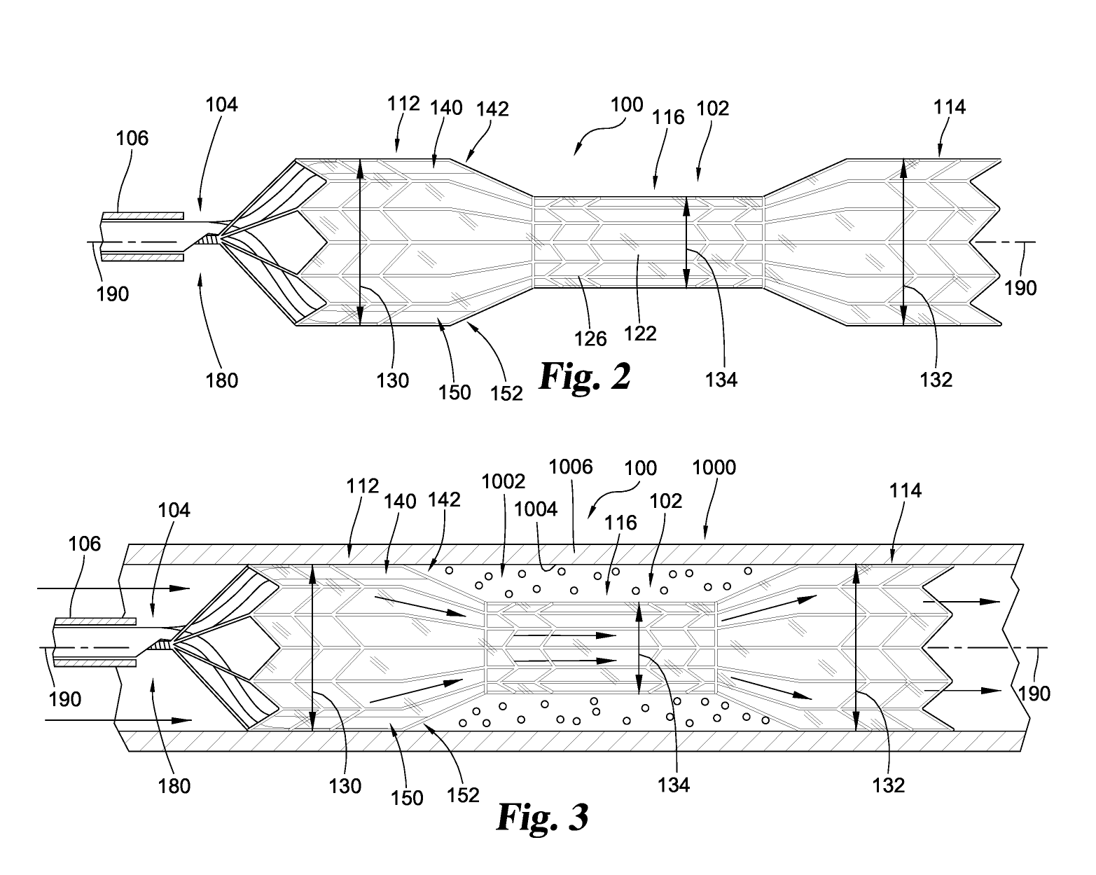

In some arrangements, the stent graft includes a transitional portion arranged to increase flexibility in the proximal end region. As illustrated in FIGS. 2-4, the transitional portion 180 can be positioned along a central, longitudinal axis 190 of the stent graft when the stent graft is an expanded configuration. In some instances, as illustrated in FIGS. 5, 6, and 7, the transitional portion includes a portion of helically-extending material (e.g., a spiral cut cannula). Advantageously, such an arrangement may aid in strain relief in the device, as taught in U.S. Publication No. 2016/0106405 and titled TRANSITIONAL GEOMETRY FOR AN EXPANDABLE MEDICAL DEVICE.

In some instances, a pushing member (e.g., a wire) and/or pulling member (e.g., a wire or string) may extend proximally from the transitional portion so as to be operable to selectively push the stent graft from a sheath and/or withdraw the stent graft into a sheath. In some instances, it is contemplated that the pushing member or pulling member may have a length sufficient to extend out of the body of the patient (e.g., a length of 30 cm or more). It is contemplated that, in some embodiments, the transitional portion may be configured and arranged to be grasped by a retrieval device, such as a retrieval loop, for the subsequent capture and withdrawal of the stent graft after deployment of the stent graft within the body of a patient.

The infusion/aspiration lumens may extend along an outward-facing surface of the transitional portion and/or along an inward-facing surface of the transitional portion. For example, in some instances, the first infusion/aspiration lumen extends through the interior of the transitional portion.

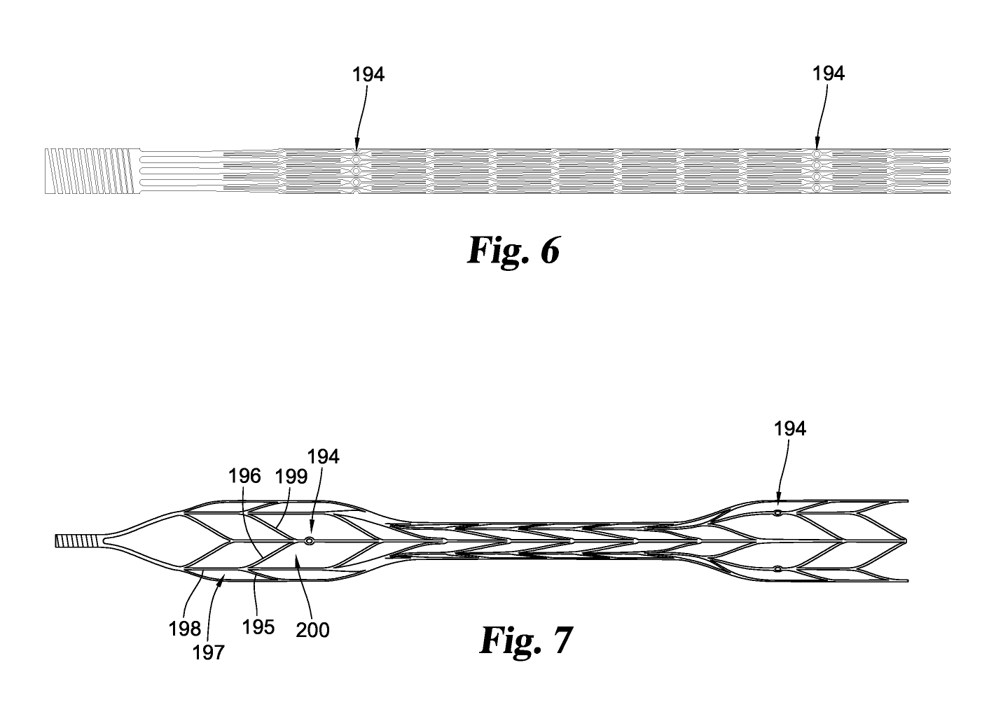

FIGS. 6 and 7 illustrate another embodiment of a stent for the stent grafts disclosed herein. FIG. 6 illustrates the stent in the contracted configuration, and FIG. 7 illustrates the stent in the expanded configuration. As will be appreciated, stents may be selected or designed to achieve a desired contracted configuration and expanded configuration.

The stent of any of the stent grafts disclosed herein may include a series of zig-zagging straight sections joined by bends, such as to form a plurality of serpentine rings. The stent of the stent graft may be formed of an integral frame or a series of discrete frames along the length of the stent graft. For example, serpentine rings may be interconnected with longitudinal structural members and/or by securement of the rings to the graft material of the stent graft. The stent may be fabricated from a cannula. In some instances, the stent may have longitudinal segments of laterally interconnected closed cells, as disclosed in U.S. Pat. Nos. 6,231,598, and 6,743,252 which are hereby incorporated by reference in their entirety.

In some embodiments, apices formed by intersecting struts and/or bends in struts of the stent are intersected by a longitudinally extending strut of the stent. For example, as shown in FIG. 7, proximal ends of strut 195 and strut 196 meet at a proximal apex 197, and apex 197 is intersected by longitudinal strut 198 that extends proximally from proximal apex 197. Similarly, distal ends of strut 196 and 199 meet at a distal apex 200 intersected by a longitudinal strut. Advantageously, having longitudinal struts intersecting and/or extending away from apices of the stent can aid in the delivery and/or retrieval of the stent graft from a sheath. For instance, longitudinal strut 198 may help pull apex 197 inward (i.e., towards the longitudinal axis of the stent) as a sheath is advanced distally along the stent, thereby preventing the apex from becoming caught on the end of the sheath as the proximal end region of the stent is contracted and withdrawn into the sheath. Additionally, having apices positioned on longitudinally-extending members (e.g., longitudinal struts) can aid in manufacturing by improving the ease by which a stent may be advanced and/or withdrawn over a mandrel.

The struts of the stent grafts described herein may define apertures for one or more markers 194 (e.g., radiographic markers and/or echogenic markers) and/or for the infusion/aspiration lumens. The markers may be arranged to indicate the position of the device or a portion of the device (e.g., the intermediate region) after the device has been inserted into the body of the patient. For example, markers may be positioned at the ends of the intermediate region and/or at the proximal end and/or distal end of the graft material. Additionally, the markers may be arranged to indicate whether the stent graft is in the expanded or contracted configuration.

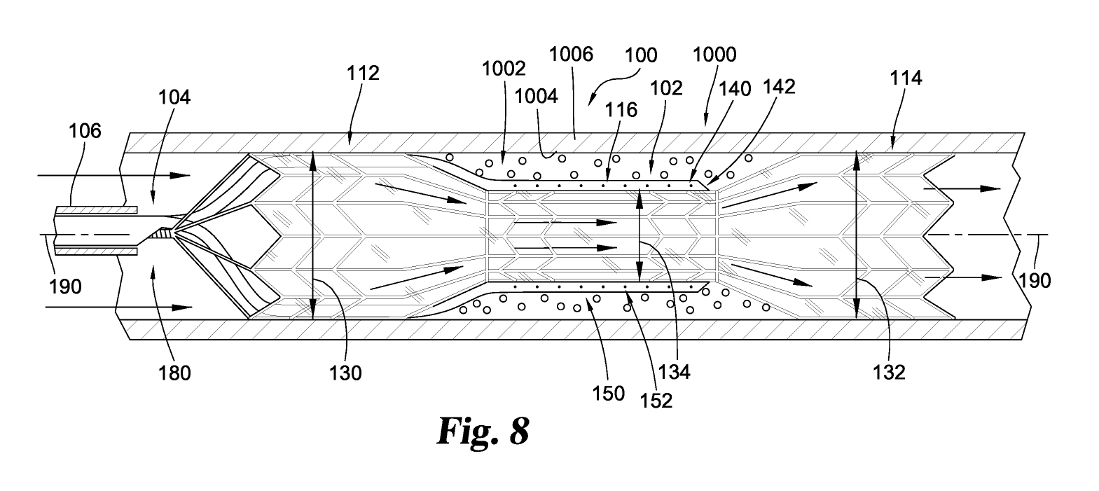

FIG. 8 illustrates the infusion/aspiration lumens extending along the intermediate region. In some, but not necessarily all embodiments, the infusion/aspiration lumens have a plurality of openings/ports for infusion/aspiration alongside the intermediate region. Such an arrangement may be used with any of the above-mentioned devices, including those in which the infusion/aspiration lumens extend along an outward-facing surface of the stent graft in the proximal end region. Similarly, as will be appreciated by those of skill in the art, any of the devices mentioned herein may have one or more lumens for infusion and one or more lumens for aspiration.

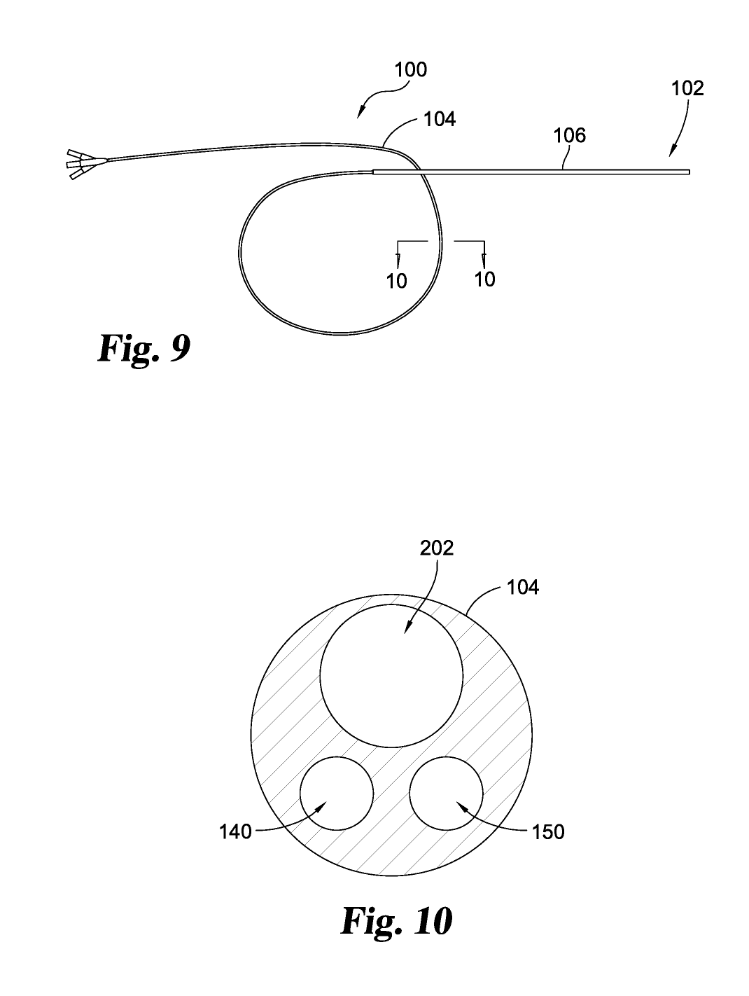

FIG. 9 illustrates the device 100 with the stent graft 102 positioned within the sheath 106. FIG. 10 illustrates a cross-sectional view of an embodiment of catheter 104. As discussed above, the catheter may include one or more lumens for the infusion and/or withdrawal of fluid to the space adjacent the intermediate region of the stent graft on the outward-facing surface of the graft material. For example, the catheter may have a first infusion/aspiration lumen 140, a second infusion/aspiration lumen 150, and a guide wire lumen 202.

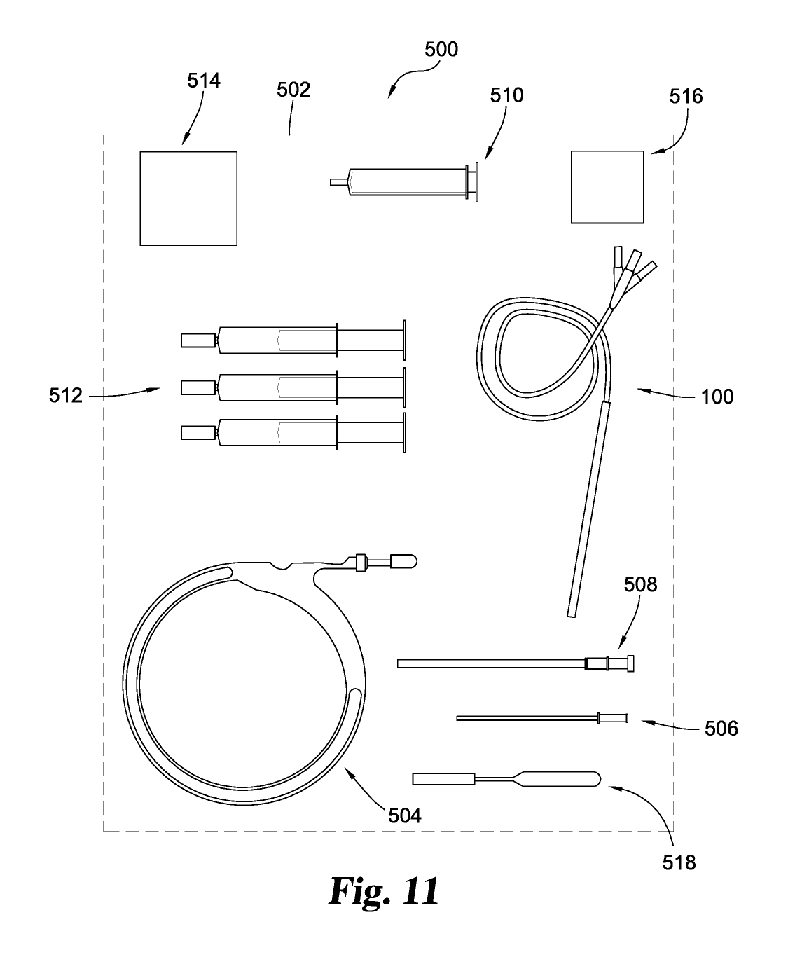

Kits (e.g., trays) containing the devices described above, and others, are contemplated. For example, as illustrated in FIG. 11, a kit 500 enclosed within sterily sealed medical package 502 may include a flexible-tip wire guide 504, an introducer needle 506, a dilator 508, an empty container (e.g., a vial or syringe) 510, a prefilled container 512, gauze sponges 514; a drape 516, a safety scalpel 518 and any of the devices mentioned above, including the devices illustrated in the accompanying figures.

A prefilled container (e.g., a vial or syringe) included in the kit may contain a therapeutic agent useful with the above described devices. For example, a prefilled vial or syringe may include small molecule drugs useful for localized chemotherapy/oncology and/or vascular intervention such as dissolving thrombus and/or reducing vascular calcification. For example, drugs such as paclitaxel, rapamycin, myotropic/neurotropic antispasmodics, and anticalcificants such as phosphate binders may be included in the prefilled vials or syringes. Additionally or alternatively, contrast agents may be included in the solution or suspension contained within the prefilled vials and syringes. Contrast agents suitable for MRI, X-Ray, and/or ultrasound imaging are all contemplated, such as gadolinium, manganese, iron oxide, and iodine-based (ionic/non-ionic) contrast agents, just to name a few non-limiting examples.

Advantageously, nanoparticles may be included in the above kits and/or delivered using the above described devices. For example, organic, inorganic, and/or complex/polymeric nanoparticles useful for thermal ablation and targeted drug-delivery are contemplated. This includes but is not limited to liposomes, micelles, perfluorocarbons, gold nanoparticles, superparamagnetic iron oxide nanoparticles (SPION), dendrimers and functionalized nanoparticles.

Similarly, macromolecules may be included in the above kits and/or delivered using the above described devices. For example, proteins, peptides, and/or synthetic polymers useful for biochemical thrombectomy, cell adhesion, coercive morphogenesis, prolonged drug-release, and/or sealants are contemplated. This includes but is not limited to fibrinolytics (e.g., urokinase, tPA), adhesional proteins (e.g., Fn, Lama, Col), growth factors (e.g., VEGF, TGF, Insulin), drug-eluting gels, and glues.

It is also contemplated that cells may be included in the above kits and/or delivered using the above described devices. For example, differentiated, stem/progenitor, and/or genetically modified cells useful for re-endothelialization, endothelial regeneration and/or cellular therapy are contemplated. This includes but is not limited to endothelial cells, mesenchymoangioblasts, and bioengineering immune cells.

Systems incorporating the devices and/or methods herein are also contemplated. For example, a system including a device disclosed herein and an apparatus for infusing/aspirating a fluid through one or more of the infusion/aspiration lumens of the device are contemplated. The apparatus or the system may include syringes and/or pumps. In some instances, the system may be configured to circulate a fluid through the infusion/aspiration lumen(s) of the device according to a treatment protocol. For example, the system may include one or more pumps configured to infuse therapeutic agent through the first infusion/aspiration lumen into the space between the graft material and the vessel wall and aspirate fluid from the same space using the second infusion/aspiration lumen. In some instances, the treatment protocol includes adjusting the inflow and outflow of therapeutic agent into a target site. This adjustment can be based on the catalytic performance of the therapeutic agent in the target site. Additionally, adjusting inflow and outflow of fluid can facilitate localization of therapeutics at the target site. It is contemplated that the treatment protocol may include adjusting the inflow of the suspension of cells and outflow of fluid (e.g., suspension of cells that did not adhere to the target site).

Various methods of using the above described devices are contemplated. As mentioned above, the above devices may be useful for localized chemotherapy/oncology, vascular intervention such as dissolving thrombus and/or for reducing vascular calcification; for injecting contrast agents suitable for MRI, X-Ray, and/or ultrasound imaging; for thermal ablation and targeted drug-delivery; for biochemical thrombectomy, cell adhesion, coercive morphogenesis, prolonged drug-release, and/or sealants; and/or for endothelialization, endothelial regeneration and/or cellular therapy. It will be contemplated, however, that the above devices may be used for other therapies as well. For example, the above devices may be positioned over a medical device (e.g., a stent) and used to “treat” the medical device (e.g., promote endothelization of the medical device). It is also contemplated that the above device may be useful for sealing vessel dissections, such as aortic dissections. Such devices and/or methods may include use of a hydrogel sealant.

In one exemplary method of using a device described herein, a distal end of a sheath containing the stent graft of the device in a contracted configuration is advanced through a vessel of a patient towards a target location within the body of a patient. In some instances, the distal end of the sheath and the stent graft are advanced percutaneously (i.e., through the skin of a patient). In other instances, the distal end of the sheath and the stent are advanced through a natural body opening (e.g., through the mouth and into the esophagus or trachea).

Once positioned at the target location within the patient, the sheath may be withdrawn and/or the stent graft advanced so as to remove the stent graft from within the sheath. After removal from the sheath, the stent graft may be configured from the contracted configuration into the expanded configuration. For self-expanding stent grafts, the stent may self-expand into the expanded configuration. Advantageously, such self-expanding stent grafts can maintain juxtaposition with vessel walls as a function of vessel wall remodeling during a therapy (i.e., constriction or dilation). For balloon-expandable stent grafts, one or more balloons positioned within the lumen of the stent graft may be expanded so as to expand the stent graft into the expanded configuration.

In the expanded configuration, a space is defined between the outward-facing surface of the graft material of the intermediate region of the stent graft and the inner surface of the body vessel. Additionally, in the expanded configuration, at least the first infusion/aspiration port of the first infusion/aspiration lumen is in fluid communication with the above-mentioned space. A fluid (e.g., a therapeutic agent in a solution or suspension and/or one or more liquid chemicals that can be formed into a hydrogel) may be infused through at least the first infusion/aspiration lumen and out of the first infusion/aspiration port into the space, causing the fluid to come into contact with the outward-facing surface of the graft material and with the inner surface of the vessel wall. Multiple fluids may be infused and/or mixed when in contact with the outward-facing surface of the graft material. In some instances, those fluids are mixed to form a hydrogel. It is also contemplated that a fluid substance may be infused into contact with the outward-facing surface of the graft material and then irradiated (e.g., irradiated with UV light) so as form a hydrogel and/or cure the infused substance or mixture.

In some instances, a fluid (e.g., blood) may be withdrawn from the space after expansion of the stent graft into the expanded configuration and before and/or during infusion of a therapeutic agent. In embodiments having a second infusion/aspiration lumen and a second infusion/aspiration port, a fluid may be withdrawn and/or infused through the second infusion/aspiration lumen before, during, and/or after the withdrawal and/or infusion of fluid through the first infusion/aspiration lumen. For example, in some instances, fluid is withdrawn from the space through the second infusion/aspiration lumen while fluid is simultaneously being infused into the space from the first infusion/aspiration lumen and first infusion/aspiration port. Advantageously, such an arrangement can allow for the removal of cells that did not adhere to the target site while supplying new viable cells.

Advantageously, the above infusion and/or withdrawal of fluid while the stent graft is in the expanded configuration may occur while body fluid is capable of flowing through the lumen of the stent graft. After infusion and/or withdrawal of fluid through the first infusion/aspiration lumen and/or second infusion/aspiration lumen, the stent graft may be selectively retrieved, such as by withdrawing the transitional portion into the sheath, contracting the stent graft into the contracted configuration, and withdrawing the stent graft and the sheath from the body of the patient. Retrieval may be accomplished with the stent graft either attached or detached from the catheter.

In some instances, the catheter is detached from the stent graft after infusion and/or withdrawal of fluid through at least the first infusion/aspiration lumen and first infusion/aspiration port. In such instances, the catheter may be detached form the stent graft and the stent graft left in place (e.g., implanted in the patient) permanently or temporarily (e.g., for a period of minutes, hours, days, weeks, or months).

Advantageously, the above devices and methods can allow for a localized procedure within a vessel of a patient without substantial occlusion of the vessel during the treatment. In some instances, the average cross-sectional area of the lumen (measured perpendicular to the longitudinal axis) defined by the intermediate region of the stent graft is at least 25% of the average cross-sectional area of the lumen defined by the proximal end region and/or distal end region. In some embodiments, the average cross-sectional area of the lumen defined by the intermediate region of the stent graft is at least 50% or at least 75% of the average cross-sectional area of the lumen defined by the proximal end region and/or distal end region. In some instances, the average outer dimension of the intermediate region may be at least 25% to 75% the average outer dimension of the proximal end region and/or the distal end region. In some embodiments, the average outer dimension of the intermediate region is approximately 50% the average outer dimension of the proximal end region and/or the distal end region.

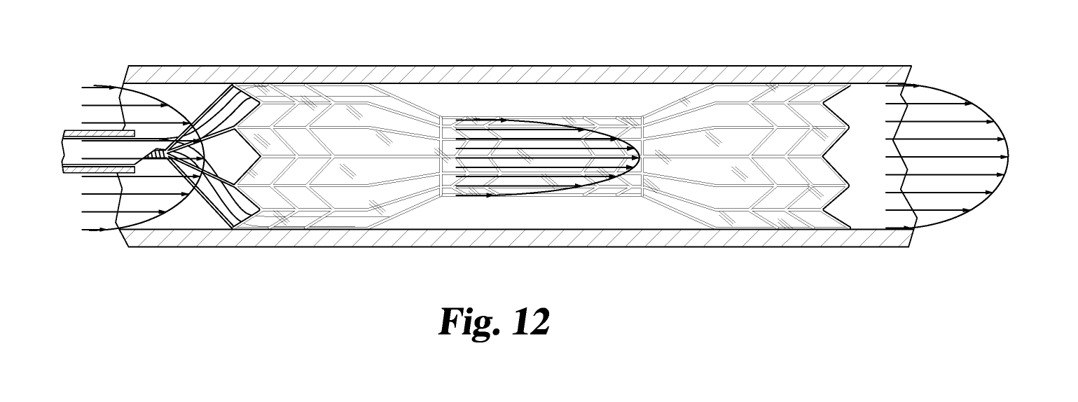

As illustrated in FIG. 12, the graft material of the stent graft separates the therapeutic agent provided to the target area from the bodily fluid flowing through the body vessel, with the therapeutic agent on one side of the graft material and the bodily fluid on the other side of the graft material. In many embodiments, in inside dimension of the stent graft will be greater in the proximal end region and/or distal end region of the stent graft than in the intermediate region, resulting in the velocity of the bodily fluid flowing through the body vessel and the stent graft, illustrated by the velocity curves in FIG. 12, to be greater in the intermediate region of the stent graft than in the proximal end region and/or distal end region.

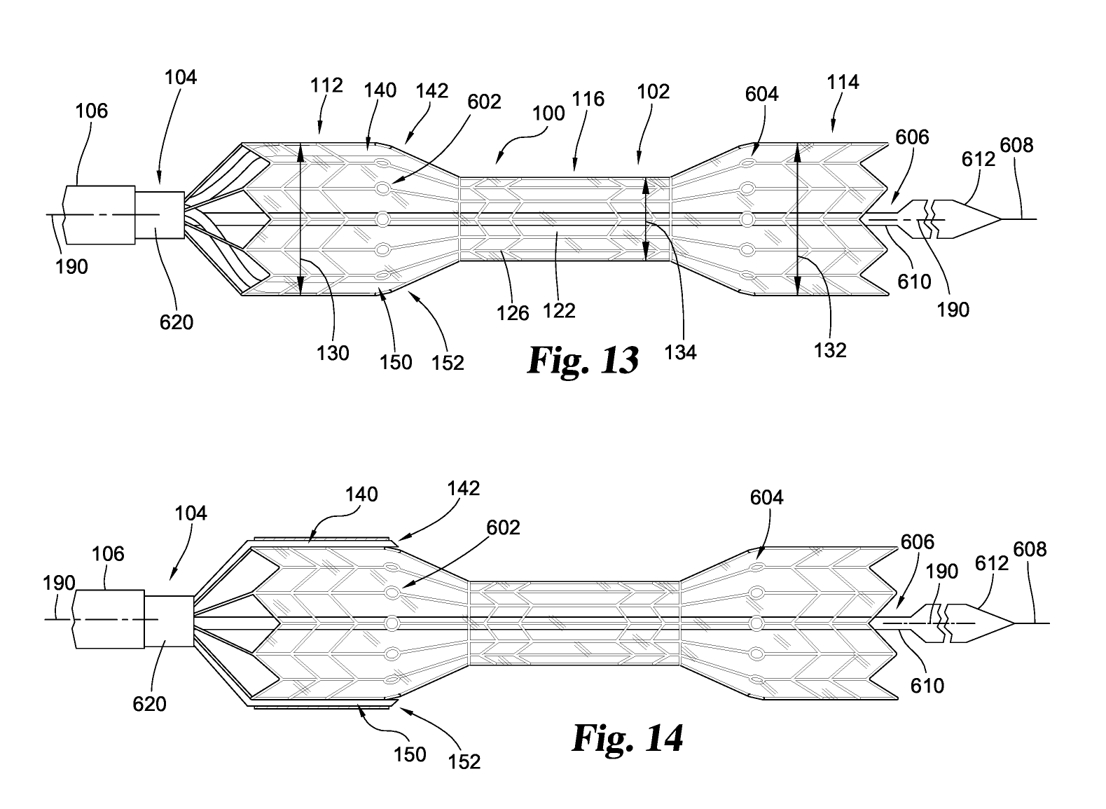

The embodiments described herein may include one or more radiopaque markers. For example, embodiments of device 100 may have a proximal radiopaque marker 602 and a distal radiopaque marker 604, as illustrated in FIGS. 13 and 14. The proximal radiopaque marker can be positioned in the proximal end region 112, and the distal radiopaque marker can be positioned in the distal end region 114.

In some instances, the proximal radiopaque marker is positioned at or near a distal end of proximal end region 112 (e.g., adjacent intermediate region 116). Similarly, in some arrangements, the distal radiopaque marker is positioned at or near a proximal end of distal end region 114 (e.g., adjacent intermediate region 116). In this way, the proximal and/or distal radiopaque markers can indicate the proximal and/or distal boundaries of the intermediate region when visualized with a medical imaging system, such as x-ray or ultrasound. Advantageously, this can indicate the area in the vessel that will receive infusate from one or more fluid infusion/aspiration lumens (e.g., first infusion/aspiration lumen 140 and/or second infusion/aspiration lumen 150) during an infusion procedure.

More or fewer radiopaque markers than those described above and illustrated in the figures are contemplated. For example, in some embodiments, device 100 can have a proximal radiopaque marker positioned at or near the proximal end of the stent and/or the graft material (e.g., covering material) in the proximal end region of the device. Similarly, the device can have a distal radiopaque marker positioned at or near the distal end of the stent and/or the graft material (e.g., covering material) in the distal end region of the device. Advantageously, such markers can indicate the sealing regions of the stent graft so as to help avoid the covering material in the proximal end region and/or distal end regions being inadvertently positioned over a branch vessel during a procedure.

Embodiments described herein may also include guidewire lumen for receiving a guidewire. The guidewire lumen may be defined by a catheter segment that extends through the central lumen of the stent graft. In some arrangements, the guidewire lumen may terminate at a distal end positioned distal of the distal end region of the stent graft. For example, the guidewire lumen may be defined by a catheter segment that extends beyond the distal-most end of the stent graft. In many instances, the catheter segment defining the guidewire lumen includes a dilator tip at the distal end.

Both FIGS. 13 and 14 illustrate a guidewire lumen 606 for receiving a guidewire 608, as described above. FIG. 13 illustrates an embodiment having an infusion/aspiration lumen positioned radially within the stent in the proximal end region of the stent graft, and FIG. 14 illustrates an embodiment having an infusion/aspiration lumen positioned radially outward of the stent in the proximal end region of the stent graft.

The catheter segment defining the guidewire lumen may be a segment of the same catheter defining the first and/or second infusion/aspiration lumens. For example, the guidewire lumen, the first infusion/aspiration lumen, and/or the second infusion/aspiration lumen may be lumens of a single catheter (e.g., a dual-lumen or tri-lumen catheter). Alternatively, the catheter segment defining the guidewire lumen may be a separate catheter from a catheter defining the first and/or second infusion/aspiration lumens.

The embodiments described herein may also include a pusher 620 arranged to push the device from the delivery sheath. The pusher may be coupled to the stent graft and/or catheter segments defining the first infusion/aspiration lumen, second infusion/aspiration lumen, and/or guidewire lumen. In many instances, the pusher is arranged to pull the stent graft into the delivery sheath.

The stent graft of any of the embodiments described herein may be a self-expanding stent graft or a balloon expandable stent graft. In some arrangements, one or more expandable balloons may be positioned inside the proximal and distal end regions of the stent graft. For example, a first expandable balloon may be positioned inside the proximal end region of the stent graft and a second expandable balloon may be positioned inside the distal end region of the stent graft. In some instances, an interior of an expandable balloon portion positioned inside the proximal end region of the stent graft is in fluid communication with an interior of an expandable balloon portion positioned inside the distal end region of the stent graft. In some instances, the portion of the central lumen within the intermediate region of the stent graft is free of an expandable balloon.

Expandable balloons for expanding portions of the stent graft are in fluid communication with one or more inflation/deflation lumens defined by one or more catheter segments. The one or more catheter segments may be a portion of a catheter defining the guidewire lumen, first infusion/aspiration lumen, and/or second infusion/aspiration lumen, or the one or more catheter segments may be a portion of a separate catheter. Advantageously, in some embodiments, the expandable balloon(s) are removable from inside the stent graft after the stent graft is expanded to the expanded configuration. For example, the expandable balloon(s) may be provided on a balloon catheter that is removably positioned in the central lumen of the stent graft.

The delivery sheath mentioned in any embodiment described herein may be a splittable sheath. Such sheaths are also referred to as “peel away” sheaths and are capable of being split along their length so as to allow removal of a catheter and/or guide wire positioned within the lumen of the sheath without movement of the sheath over an end of the catheter and/or guidewire.

The following numbered clauses set out specific embodiments that may be useful in understanding the present invention:

1. A device, comprising:

a stent graft extending from a proximal end region to a distal end region and having an intermediate region positioned intermediate said proximal end region and said distal end region;
said stent graft configurable from a contracted configuration to an expanded configuration;
said stent graft defining a central lumen extending from the proximal end region to the distal end region in said expanded configuration and having a graft material portion in said intermediate region; and
said graft material portion having an inward-facing side that faces towards said central lumen and an outward-facing side that faces away from said central lumen; and

a first infusion/aspiration lumen extending along said proximal end region and in fluid communication with a first infusion/aspiration port that opens to said outward-facing side of said graft material; the device further characterized by:
(I) the first infusion/aspiration lumen extending along an outward-facing side of a graft material of said stent graft in said proximal end region; and/or
(II) said proximal end region and said distal end region each having an average outer dimension when said stent graft is in said expanded configuration, said average outer dimensions, in said expanded configuration, of said proximal end region and said distal end region each being greater than an average outer dimension defined by said graft material of said intermediate region in said expanded configuration.
   2. The device of clause 1, wherein said stent graft includes a stent having an outward-facing side that faces away from the central lumen of the stent graft and an inward-facing side that faces towards the central lumen; and

wherein said first infusion/aspiration lumen extends along said inward-facing side of said stent in said proximal end region.

3. The device of any preceding clause, wherein said stent is self-expanding.
4. The device of any preceding clause, wherein a second infusion/aspiration port is in fluid communication with a second infusion/aspiration lumen; and

wherein said second infusion/aspiration port opens to said outward-facing side of said graft material.

5. The device of clause 4, wherein said infusion/aspiration ports are spread evenly around said intermediate region of said stent graft.
6. The device of any preceding clause, wherein said first infusion/aspiration lumen is defined by a catheter.
7. The device of any preceding clause, wherein said first infusion/aspiration lumen is positioned between said graft material and a second graft material; and

wherein said second graft material is bonded to said graft material.

8. The device of any preceding clause, wherein said infusion/aspiration lumen extends in a proximal direction through a sheath; and

wherein said sheath is sized and configured to contain said stent graft in said contracted configuration.

9. The device of any preceding clause, wherein said average outer dimensions in said expanded configuration are said average outer dimensions when said stent graft is expanded in its unconstrained condition.
10. The device of any preceding clause, wherein said proximal end region, said intermediate region, and said distal end region each have an average outer dimension in said contracted configuration; and

wherein said average outer dimension of said proximal end region, said intermediate region, or said distal end region in said expanded configuration is at least 20% greater than the average outer dimensions of said same region in said contracted configuration.

11. The device of any preceding clause, wherein said proximal end region, said intermediate region, and said distal end region each have an average outer dimension in said contracted configuration of 7 mm or less.
12. The device of any preceding clause, wherein said proximal end region of stent graft includes a transitional portion; and

wherein said transitional portion has a portion positioned along a central, longitudinal axis of said stent graft when said stent graft is in an expanded configuration.

13. The device of any preceding clause, wherein the transitional portion includes a helically-extending material.
14. The device of any preceding clause, wherein proximal end region of the stent graft is free of apices not having struts extending proximally therefrom.
15. A method of using the device of any preceding clause, comprising:

infusing a therapeutic agent through said first infusion/aspiration lumen towards said first infusion/aspiration port and out of said first infusion/aspiration port into contact with said outward-facing side of said graft material.

16. The method of clause 15 or a method of using the device of any of clauses 1-14, comprising:

drawing a fluid contacting said outward-facing side of said graft material through the first infusion/aspiration port and into the first infusion/aspiration lumen.

17. The method of clause 15 or clause 16 or a method of using the device of any of clauses 1-14, comprising:

drawing a fluid contacting said outward-facing side of said graft material through a second infusion/aspiration port and into a second infusion/aspiration lumen;

wherein said second infusion/aspiration port in opens to said outward-facing side of said graft material.

18. A method of using the device of any of clauses 1-14, comprising:

infusing a first substance in liquid form through said first infusion/aspiration lumen towards said first infusion/aspiration port and out of said first infusion/aspiration port into contact with said outward-facing side of said graft material; and

transitioning said first substance from liquid form into hydrogel form.

19. The method of clause 18, comprising:

infusing a cross-linker through a second infusion/aspiration lumen towards a second infusion/aspiration port and out of said second infusion/aspiration port into contact with said outward-facing side of said graft material so as to mix the cross-linker with the first substance while in contact with said outward-facing side of said graft material.

20. A kit, comprising:

a stent graft, a catheter, and a sheath within a sterily sealed package;

wherein said stent graft has a proximal portion, a distal portion, an intermediate portion positioned intermediate said proximal portion and said distal portion, and a stent configurable from a contracted configuration to an expanded configuration;

wherein said stent graft includes a graft material portion extending along said intermediate portion;

wherein said proximal portion and said distal portion each have an average outer dimension greater than an average outer dimension defined by said graft material portion in said intermediate portion;

wherein said catheter communicates with a first infusion/aspiration port through a first infusion/aspiration lumen;

wherein said first infusion/aspiration port opens to an outer side of said graft material of said stent graft; and

wherein said stent graft and said catheter are positioned within a lumen of said sheath with said stent in said contracted configuration.

21. The kit of clause 20, further comprising:

a therapeutic agent in a container within said sterily sealed package.

22. A device, comprising:

a stent graft defining a central lumen extending from a proximal end region to a distal end region, said stent graft having an intermediate region positioned intermediate said proximal end region and said distal end region; and

a first infusion/aspiration lumen extending along an outward-facing side of a graft material of said stent graft in said proximal end region and communicating with a first infusion/aspiration port is positioned in said intermediate region.

23. The device of clause 22, wherein the stent graft includes a dog-bone shaped stent.
24. The device or method of any one of clauses 1-19 or 22-23, comprising a catheter segment extending through the proximal end region, intermediate region, and distal end region of the stent graft and defining a guidewire lumen.
25. The kit of clause 20 or 21, comprising a catheter segment extending through the proximal portion, intermediate portion, and distal portion of the stent graft and defining a guidewire lumen

1. A device, comprising:
a stent graft extending from a proximal end region to a distal end region and having an intermediate region positioned intermediate said proximal end region and said distal end region;
said stent graft configurable from a contracted configuration to an expanded configuration;
said stent graft defining a central lumen extending from the proximal end region to the distal end region in said expanded configuration and having a graft material portion in said intermediate region;
said graft material portion having an inward-facing side that faces towards said central lumen and an outward-facing side that faces away from said central lumen;
said proximal end region and said distal end region each having an average outer dimension when said stent graft is in said expanded configuration;
said average outer dimensions, in said expanded configuration, of said proximal end region and said distal end region each being greater than an average outer dimension defined by said graft material of said intermediate region in said expanded configuration; and
a first infusion/aspiration port in fluid communication with a first infusion/aspiration lumen;
said first infusion/aspiration port opening on said outward-facing side of said graft material and said first infusion/aspiration lumen extending proximally from said first infusion/aspiration port along said proximal end region of said stent graft.

2. The device of claim 1, wherein said stent graft includes a stent having an outward-facing side that faces away from the central lumen of the stent graft and an inward-facing side that faces towards the central lumen; and
wherein said first infusion/aspiration lumen extends along said inward-facing side of said stent in said proximal end region.

3. The device of claim 2, wherein said stent is self-expanding.

4. The device of claim 1, wherein a second infusion/aspiration port is in fluid communication with a second infusion/aspiration lumen; and
wherein said second infusion/aspiration port opens to said outward-facing side of said graft material.

5. The device of claim 4, wherein said infusion/aspiration ports are spread evenly around said intermediate region of said stent graft.

6. The device of claim 1, wherein said first infusion/aspiration lumen is defined by a catheter.

7. The device of claim 1, wherein said first infusion/aspiration lumen is positioned between said graft material and a second graft material; and
wherein said second graft material is bonded to said graft material.

8. The device of claim 1, wherein said infusion/aspiration lumen extends in a proximal direction through a sheath; and
wherein said sheath is sized and configured to contain said stent graft in said contracted configuration.

9. The device of claim 1, wherein said average outer dimensions in said expanded configuration are said average outer dimensions when said stent graft is expanded in its unconstrained condition.

10. The device of claim 1, wherein said proximal end region, said intermediate region, and said distal end region each have an average outer dimension in said contracted configuration; and
wherein said average outer dimension of said proximal end region, said intermediate region, or said distal end region in said expanded configuration is at least 20% greater than the average outer dimensions of said same region in said contracted configuration.

11. The device of claim 10, wherein said proximal end region, said intermediate region, and said distal end region each have an average outer dimension in said contracted configuration of 7 mm or less.

12. The device of claim 1, wherein said proximal end region of stent graft includes a transitional portion; and
wherein said transitional portion has a portion positioned along a central, longitudinal axis of said stent graft when said stent graft is in an expanded configuration.

13. The device of claim 12, wherein the transitional portion includes a helically-extending material.

14. The device of claim 1, wherein proximal end region of the stent graft is free of apices not having struts extending proximally therefrom.

15. A method of using the device of claim 1, comprising:
infusing a therapeutic agent through said first infusion/aspiration lumen towards said first infusion/aspiration port and out of said first infusion/aspiration port into contact with said outward-facing side of said graft material.

16. The method of using the device of claim 1, comprising:
drawing a fluid contacting said outward-facing side of said graft material through the first infusion/aspiration port and into the first infusion/aspiration lumen.

17. The method of claim 15, further comprising:
drawing a fluid contacting said outward-facing side of said graft material through a second infusion/aspiration port and into a second infusion/aspiration lumen;
wherein said second infusion/aspiration port in opens to said outward-facing side of said graft material.

18. A method of using the device of claim 1, comprising:
infusing a first substance in liquid form through said first infusion/aspiration lumen towards said first infusion/aspiration port and out of said first infusion/aspiration port into contact with said outward-facing side of said graft material; and
transitioning said first substance from liquid form into hydrogel form.

19. The method of claim 18, comprising:
infusing a cross-linker through a second infusion/aspiration lumen towards a second infusion/aspiration port and out of said second infusion/aspiration port into contact with said outward-facing side of said graft material so as to mix the cross-linker with the first substance while in contact with said outward-facing side of said graft material.

20. A kit, comprising:
a stent graft, a catheter, and a sheath within a sterily sealed package;
wherein said stent graft has a proximal portion, a distal portion, an intermediate portion positioned intermediate said proximal portion and said distal portion, and a stent configurable from a contracted configuration to an expanded configuration;
wherein said stent graft includes a graft material portion extending along said intermediate portion;
wherein said proximal portion and said distal portion each have an average outer dimension greater than an average outer dimension defined by said graft material portion in said intermediate portion;
wherein said catheter communicates with a first infusion/aspiration port through a first infusion/aspiration lumen;
wherein said first infusion/aspiration port opens to an outer side of said graft material of said stent graft; and
wherein said stent graft and said catheter are positioned within a lumen of said sheath with said stent in said contracted configuration.
